(19) United States
(12) Patent Application Publication
(10) Pub. No.: US 2026/0367035 A1
(43) Pub. Date: Nov. 19, 2026

(54) ELECTROCHEMICAL ANALYSIS OF TRACE METAL IMPURITIES

(71) Applicant: Palintest Limited, Gateshead (GB)

(72) Inventors: Simon Richard Johnston, Gateshead (GB); Steven William Birch, Gateshead (GB); Samantha Lunn, Gateshead (GB); James Iacobini, Gateshead (GB); Joseph Paul Johnston, Gateshead (GB); Mark Anthony Hutchinson, Gateshead (GB)

(21) Appl. No.: 19/155,193

(22) PCT Filed: Feb. 7, 2024
(86) PCT No.: PCT/GB2024/050330

§ 371 Date: Aug. 8, 2025

(30) Foreign Application Priority Data
Feb. 10, 2023 (GB) 2301930.0

(51) Int. Cl. G01N 27/42; B01D 15/36; G01N 27/27; G01N 27/48

(52) U.S. Cl. CPC G01N 27/42; B01D 15/362; G01N 27/27; G01N 27/48

(57) ABSTRACT

An electrochemical system, apparatus and method for the quantitative concentration analysis of target analyte ion species (such as Arsen (III) or Arsen (V) lead, cadmium or copper) in a liquid sample. The system provides a convenient, reliable and accurate means of target analyte concentration determination using voltammetric analysis as an analytical technique. An electrochemical instrument and sensor comprising a working, reference and counter electrode are configured to perform anodic stripping voltammetry (ASV) and to determine target analyte species over a large range of concentrations. The present system provides apparatus and method to normalise the resulting peak current using an initial plating/precondition current by applying at least one mathematical operation on said peak current involving a division of the peak current by the sum of charge or current measured during electrodeposition.

### FIELD OF INVENTION

The present invention relates to an electrochemical method and apparatus for the analysis of a target analyte in a liquid sample and in particular, although not exclusively, to an electrochemical analysis system for the quantitative concentration determination of electrochemically active species within an aqueous sample.

### BACKGROUND

Anodic stripping voltammetry (ASV) and potentiometric stripping analysis (PSA) are used widely for trace metal analysis within aqueous samples. ASV typically involves electrodeposition of a target metal analyte onto a working electrode with this initial stage referred to as plating or preconcentration. This is then followed by anodically re-oxidising (stripping) the deposited metal. The resulting Faradaic current as a function of voltage is then analysed as part of the ASV.

Typically, an electrochemical analysis system comprises a potentiostat capable of generating the required plating and stripping potential patterns to electrodes immersed in the sample solution that contains a target analyte. The potentiostat also measures the resulting current generated by the analyte oxidation. The potential-current relationship processed by the potentiostat is then used to determine analyte concentration. WO 00/67011 A1 discloses an electroanalytical potentiostat capable of performing voltammetric analysis.

US 2006/0011474 A1 describes a device for detecting an analyte in a liquid sample having a sensor with multiple electrodes insulated from one another on a non-conductive plate and immersible in a liquid sample, with a working electrode including an analyte-specific coating. Electrical conductors provide electrical connectivity of the sensor to a potentiostat.

Arsenic is a highly toxic substance, particularly in its inorganic forms, with long-term exposure causing serious health conditions. It is naturally present in parts of the world, with contaminated ground water being of the greatest risk to human health. According to the World Health Organization (WHO), the current recommended limit on arsenic in drinking water is 10 ppb. Despite this, many people around the world only have access to water with arsenic concentrations many times greater than 10 ppb. Accordingly, a variety of methods exist for the quantification of arsenic in drinking water and differ in their speed of execution, accuracy, cost and portability. Some of the more common methods include:
Standard method 3500-As B Silver Diethyldithiocarbamate
Electrothermal atomic absorption spectroscopy
Hydride generation atomic absorption spectroscopy
Inductively coupled plasma (ICP) emission spectroscopy
ICP-mass spectroscopy

In method 3500-As B, As(III) (arsenite) is selectively reduced to arsine gas (AsH₃) by sodium borohydride in a solution at pH 6. Arsine gas is then carried away from the reduction vessel by a stream of oxygen-free nitrogen to react with silver diethyldithiocarbamate. A red colour then develops that may be measured at a wavelength of 520 nm. As(V) (arsenate) is reduced in a similar manner although the sample must first be reduced to pH 1 by addition of HCl. As(III) will also react under these conditions and therefore must be removed beforehand, alternatively this latter reaction will give the total inorganic arsenic present. Accordingly, such methods require the use of cumbersome apparatus and produce arsine gas which is highly toxic.

Compton et al showed that a glassy carbon electrode modified with AuNPs is capable of detecting As(III) using ASV (Anal. Chem. 2004, 76, 19, 5924-5929), a silver electrode may also be used to detect As(III) (Simm, A., Banks, C. and Compton, R. (2005), The Electrochemical Detection of Arsenic (III) at a Silver Electrode. Electroanalysis, 17:1727-1733).

US 2020/0319131 A1 describes a method for detecting As(III) in water samples. A colloidal solution of AuNPs is deposited onto a carbon electrode and ASV is used to measure As(III) with a Limit Of Quantification (LOQ) of 0.075 ppb.

However, existing electrochemical methods have a number of disadvantages regarding ease of use, portability and capability to reliably and repeatably measure a large concentration range of target analyte species.

### SUMMARY OF THE INVENTION

It is an objective to provide electrochemical apparatus and methods for the convenient, reliable and repeatable measurement of a large range of target analyte concentrations within a liquid sample and preferably an aqueous sample. It is a further specific objective to provide electrochemical apparatus and methods for the quantitative concentration analysis and determination of a range of target analytes including in particular arsenic, lead, cadmium and copper. It is a further specific objective to provide an electrochemical analysis system that is portable and capable of analyte concentration analysis over a wide range of analyte concentrations. It is a further objective to provide an electrochemical analysis system configurable for use with electrochemical stripping voltammetry for the high accuracy concentration determination of target analytes in a sample liquid.

The objectives are achieved via the present apparatus and method of analysis of metal analytes in a liquid sample using electrochemical stripping voltammetry in which a peak current obtained during the oxidation of a target analyte is normalised by current data obtained during an initial plating of a working electrode with the target analyte. Accordingly, a highly sensitive electrochemical analytical system is provided with enhanced precision of target analyte concentration determination relative to existing techniques. The present apparatus and method is particularly suited for the analysis of target metal analyte species comprising any one of arsenic, lead, cadmium and copper.

According to a first aspect of the present concept there is provided an electrochemical method for the analysis of a analyte in a liquid sample comprising: performing electrochemical stripping voltammetry on a liquid sample using a potentiostat and a cell with working, reference and counter electrodes involving electrodeposition followed by oxidation of the analyte at the working electrode and measurement of current to determine an analyte peak current during the oxidation of the analyte; measuring a charge or current at the working electrode during the electrodeposition of the analyte at the working electrode; and normalising the peak current determined during the oxidation of the analyte by applying at least one mathematical operation on said peak current involving a division of the peak current by the charge or current measured during electrodeposition.

Optionally, the step of performing electrochemical stripping voltammetry comprises applying a plating potential to the working electrode and acquiring plating current data measured at the working electrode during the application of the plating potential. Optionally, the method comprises storing the plating current data at a data storage utility. Optionally, the method comprises applying a stripping potential to the working electrode and acquiring stripping current data measured at the working electrode during the application of the stripping potential. Optionally, the method comprises storing the stripping current data at a data storage utility.

Optionally, the step of applying the mathematical operation consists of division of the peak current of the oxidation of the analyte by the charge or current measured during electrodeposition.

Optionally, the analysis comprises the quantitative concentration analysis of the analyte in the liquid sample via an application of a mathematical function module to the normalised peak current to generate a normalised concentration of the analyte in the sample. Optionally, the mathematical function module is generated from a mathematical fitting of known analyte concentration data and corresponding reference peak current data measured using electrochemical stripping voltammetry during a temperature-concentration-current calibration process.

Optionally, the peak current reference data may be normalised comprising the step of during the electrodeposition of the analyte at the working electrode, measuring a charge or current at the working electrode and normalising the peak current determined during the oxidation of the analyte by dividing the peak current by the charge or current measured during electrodeposition.

Optionally, prior to the step of performing the electrochemical stripping voltammetry, removing metal cation interferants from the sample involving contacting the sample with a cation exchange component. Optionally the liquid sample is passed-through a cation exchange unit or membrane. Preferably, the cation exchange component comprises a cation exchange column and the step of contacting the sample comprises passing the sample through the cation exchange column and collecting the filtered sample.

Optionally, the method may further comprise: measuring a temperature of the sample at least during the oxidation of the analyte; and applying the measured temperature and the peak current to a mathematical function module derived from mathematical fitting of reference peak current data, reference temperature data and reference analyte concentration data measured and determined during electrochemical stripping voltammetry as part of a temperature-concentration-current calibration process to determine a temperature calibrated concentration of the analyte in the sample. Optionally, the method may comprise measuring a temperature of the sample during the process of plating and stripping and determining an average temperature.

Optionally, the temperature-concentration-current calibration process comprises measuring the temperature of a reference sample containing a known concentration of analyte simultaneously with the measuring of the charge.

Optionally, the analyte comprises a metal analyte. Optionally the metal analyte comprises any one of: arsenic, lead, cadmium and copper.

Optionally, the electrochemical stripping voltammetry comprises adsorptive stripping voltammetry and any one of anodic and cathodic voltammetry.

According to a second aspect of the present concept there is provided apparatus for the electrochemical analysis of a analyte in a sample comprising: a sensor having working, reference and counter electrodes configured to be at least partially submerged in an electrolyte solution containing a analyte; a potentiostat electrically connectable and configured to apply a potential to the working electrode; and a control utility having a normalisation module to receive charge or current data measured at the working electrode during an electrodeposition of the analyte at the working electrode and to receive current data measured during an oxidation of the analyte at the working electrode and to calculate a normalised peak current of the oxidation of the analyte at the working electrode.

Within this specification, reference to a potential applied to the working electrode encompasses the application of a potential difference between a working and another electrode such as a counter, auxiliary and/or reference electrode.

Optionally, the apparatus further comprises an electrochemical potential module to generate and apply the potential to the working electrode. Optionally, the electrochemical potential module comprises a plating module to generate and apply a plating potential to the working electrode and deposit analyte at the working electrode. Optionally, the electrochemical potential module comprises a stripping module to generate and apply a pulsed potential to the working electrode and to oxidise the analyte at the working electrode.

Optionally, the apparatus further comprises a data acquisition module to receive charge or current data during an electrodeposition and oxidation of the analyte at the working electrode.

Optionally, the normalisation module is configured to perform a mathematical operation on the peak current data acquired during oxidation of the analyte at the working electrode comprising division on the peak current data by the charge or current data acquired during electrodeposition of the analyte at the working electrode to generate the normalised peak current of oxidation of the analyte at the working electrode.

Optionally, the apparatus further comprises a mathematical function module configured to process the normalised peak current data and to calculate analyte concentration.

### BRIEF DESCRIPTION OF DRAWINGS

A specific implementation of the present invention will now be described, by way of example only, and with reference to the accompanying drawings in which:

FIG. 1 is a schematic illustration of the physical and electronic architecture of an electrochemical system configured for voltammetric analysis of target analyte species within an aqueous sample according to a specific implementation of the present concept;

FIG. 2 is a schematic illustration of a control utility part and data libraries of a potentiostat forming part of the electrochemical system of FIG. 1;

FIG. 3 is a plan view of an electrochemical sensor comprising a working, reference and counter electrode according to a specific implementation;

FIG. 4 is a flow diagram detailing various operational steps as part of an electrochemical concentration analysis of a target analyte within an aqueous sample;

FIG. 5 is a schematic illustration of a potential profile as part of differential pulse voltammetry (DPV) to oxidise target metal analyte species at a working electrode;

FIG. 6 is a graph of current response during an initial plating of a working electrode with As(III);

FIG. 7 is a graph of current response during an oxidation or stripping stage in which As(0) is oxidised at the working electrode;

FIG. 8 is a graph of differential current versus potential for a range of relatively low concentrations of As(III) at 20 °C;

FIG. 9 is a graph of peak current versus As(III) concentration for the range of As(III) concentrations of FIG. 8 at 20 °C;

FIG. 10 is a graph of differential current versus potential for a range of relatively high concentrations of As(III) at 20 °C;

FIG. 11 is a graph of peak current versus As(III) concentration for the range of As(III) concentrations of FIG. 10 at 20 °C;

FIG. 12 is a graph of differential current versus potential measured during differential pulse voltammetry (DPV) for total arsenic at 20 ppb at three different temperatures;

FIG. 13 is a 3D calibration curve of peak current, temperature and arsenic concentration suitable for temperature calibration/compensation of electrochemical concentration analysis of target analytes;

FIG. 14 is a graph illustrating the effects of cation exchange pre-processing of an aqueous sample containing a target analyte prior to DPV;

FIG. 15 is a schematic illustration of the stages of stripping voltammetry for concentration determination analysis of As(III) and total As(III plus V) according to a specific implementation of the present concept.

### DETAILED DESCRIPTION OF PREFERRED EMBODIMENT OF THE INVENTION

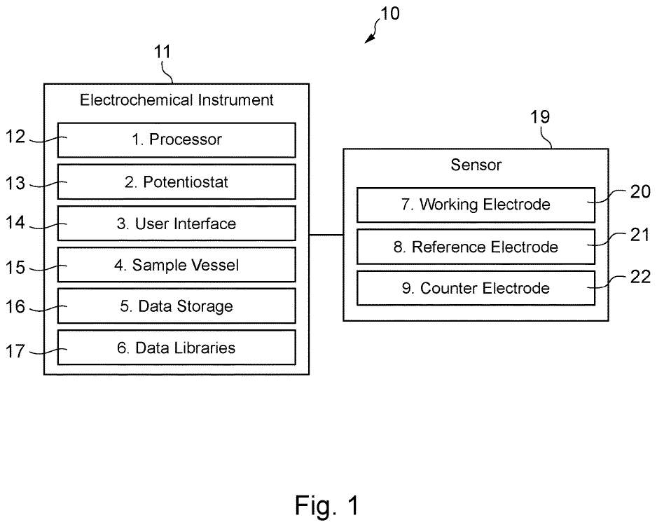

The present electrochemical system comprises apparatus and methods for the convenient, reliable and highly accurate measurement of a large range of concentrations of target analyte species. Referring to FIG. 1, the electrochemical apparatus 10 comprises generally an electrochemical instrument 11 and an electrically connectable sensor 19. Instrument 11 comprises a central processor 12 to perform computations and firmware driven operations; a potentiostat 13 to perform electrochemical testing in particular anodic stripping voltammetry (ASV) in which a desired potential is maintained between a working and reference electrode; a user interface 14; a sample vessel 15 in which test samples may be placed and tested; a data storage 16 for test and other data; data libraries 17 including calibration information for sensors loaded for accurate sensor response.

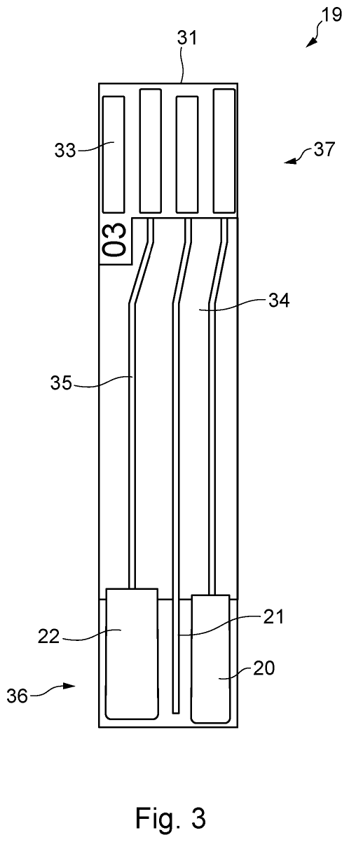

As detailed in FIG. 3, electrochemical sensor 19 comprises a screen-printed carbon working electrode 20 dosed with suitable reagent for specific electrochemical tests; a screen-printed silver electrode 21 to maintain a stable reference potential; and a screen printed counter electrode 22 to provide a current path between itself and working electrode 20 and to ensure no appreciable current flows through reference electrode 21.

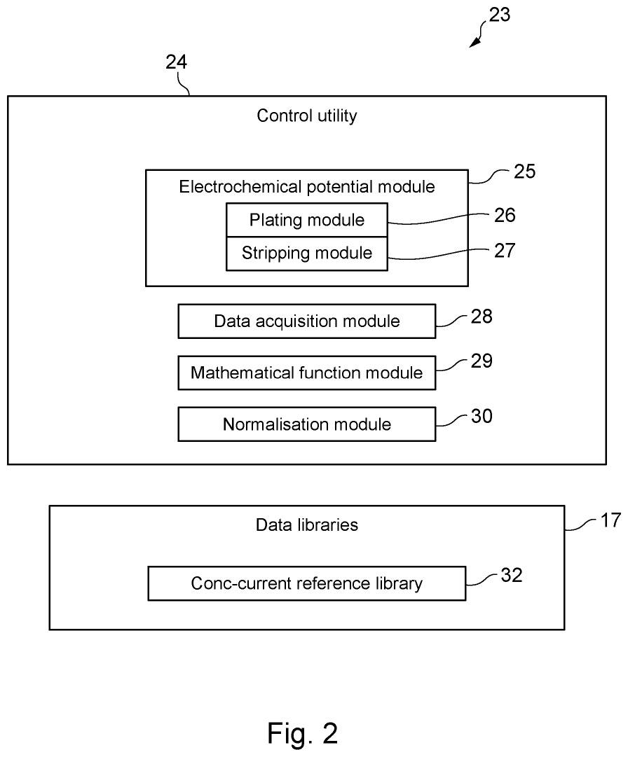

FIG. 2 illustrates schematically selected firmware 23 for potentiostat 13 comprising a control utility 24 which in turns comprises an electrochemical potential module 25 (having in turn a plating module 26 and stripping module 27); a data acquisition module 28; a mathematical function module 29 and a normalisation module 30. The data libraries 17 comprise, in particular, a concentration-current reference library 32 comprising data and coefficients derived/generated from a mathematical function correlating a range of target analyte concentrations and observed stripping peak currents generated by the ASV.

Referring to FIG. 3, sensor 19 comprises an electrically inert plastic substrate 31 having an approximate thickness of around 500 μm. Sensor 19 further comprises carbon electrical contacts 33 provided towards a first end 37 of the generally elongate sensor 19 to provide electrical connection to potentiostat 13. An insulating layer 34 covers conductive tracks 35 to enable sensor 19 to be submerged in a liquid sample. Tracks 35 comprise silver or a silver-based material to provide low resistance current pathways connecting electrical contacts 33 to an array of electrodes provided at a second end 36 of sensor 19. In particular, sensor 19 comprises a carbon counter electrode 22 to allow passage of current between itself and a working electrode 20 and to ensure substantially no current flows into reference electrode 21. Reference electrode 21 comprises silver or a silver-based material to provide a stable potential from which other potentials are applied. Working electrode 20 comprises carbon augmented/coated with gold nanoparticles. The working electrode 20 provides the working/functioning electrode where reduction and subsequent oxidation of a target metal analyte species occurs to provide the resulting electrochemical current that is measured and analysed to determine target species concentration in the liquid sample.

### Example 1

Anodic stripping voltammetry (ASV) was used in combination with a gold nanoparticle (AuNP) modified screen-printed carbon electrode (SPCE) 20. Potentiostat 13, was a Kemio™ (Palintest Ltd.). The sensor 19 was manufactured by drop-casting a solution of citrate capped AuNPs with an approximate mean diameter of 10 nm (BBI solutions, UK) on to the working electrode 20 followed by subsequent drying in an oven.

To perform Anodic Stripping Voltammetry (ASV), firstly a potential was applied between the working electrode (WE) 20 and reference electrode (RE) 21 to cause As(III) to undergo reduction to As(0), such that elemental arsenic is deposited onto AuNPs at the WE 20. This provides an initial plating or preconcentration, with different plating times allowing for different levels of sensitivity. After a predetermined amount of time elapsed, DPV was used to oxidise elemental arsenic (previously plated onto the gold coated working electrode 20 during the precondition stage). Upon oxidation, arsenic re-dissolved which allowed for measurement with high sensitivity.

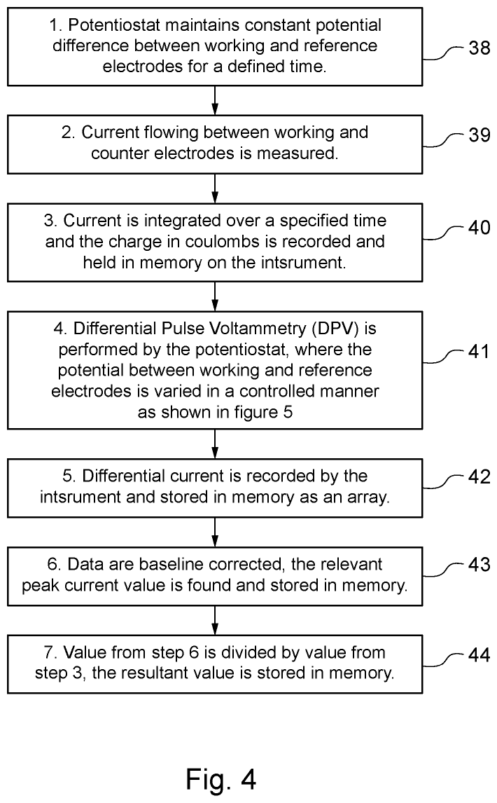

Referring to FIG. 4, at an initial stage 38, potentiostat 13 maintained a constant potential difference between working electrode 20 and reference electrode 21 for a predefined time. At stage 39, current flowing between working electrode 20 and counter electrode 22 was measured. At stage 40, the current was summed over a specified time and the total current recorded and stored within data storage 16 at instrument 11.

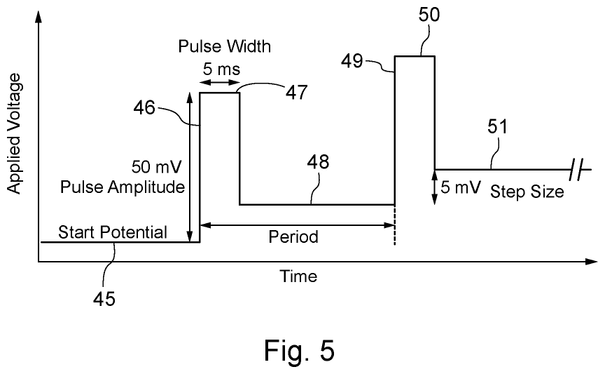

DPV was then performed by potentiostat 13 at stage 41 where the potential difference between working electrode 20 and reference electrode 21 was varied in a controlled manner as illustrated in FIG. 5. In particular, a start potential 45 was applied with typical parameters (scan rate, pulse amplitude, step size, start potential, end potential, pulse width). In particular and for completeness, a pulse amplitude was introduced 46, a pulse width 47 was maintained for a predefined time, the amplitude was then reduced and maintained for a period 48. The amplitude was then increased again 49 and maintained for a predefined pulse width 50 before the amplitude was reduced again and maintained for a predefined step size 51. It should be noted that the values of FIG. 5 (50 mV, 5 ms) are ‘typical’ values for this technique, but not necessarily what are used.

Referring again to FIG. 4, differential current was recorded by instrument 11 and stored in memory/data storage 16 as a data array at stage 42. At stage 43, the data set was baseline corrected and the relevant peak current value obtained and stored in data storage 16 at stage 43. According to a final normalisation stage 44, the peak current value obtained at stage 43 was divided by the plating current measured at stage 40 with the resulting normalised value stored within the instrument memory/data storage 16.

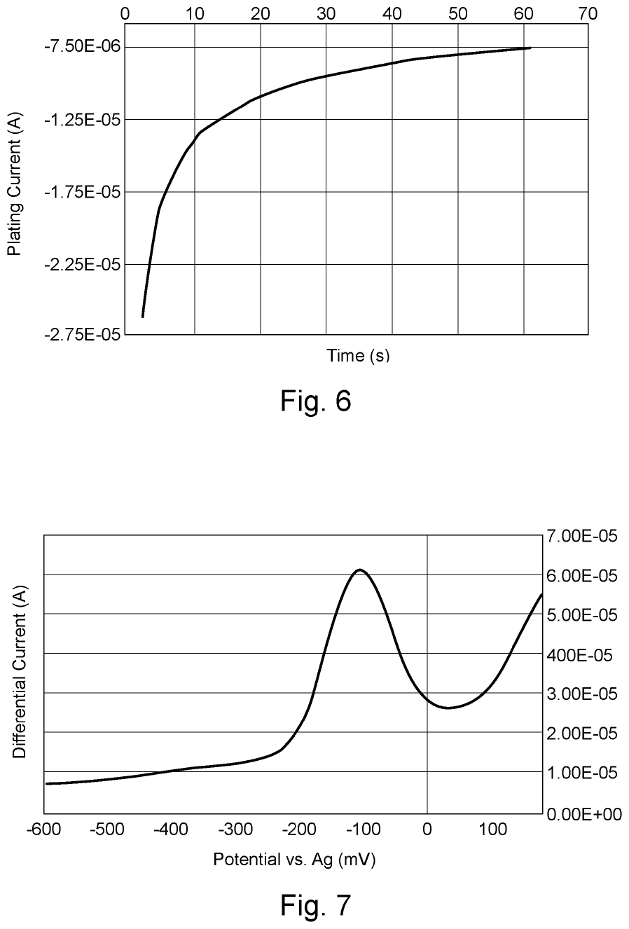

FIGS. 6 and 7 illustrate the current response for plating and stripping stages respectively where potentials are stated relative to the silver reference electrode 21.

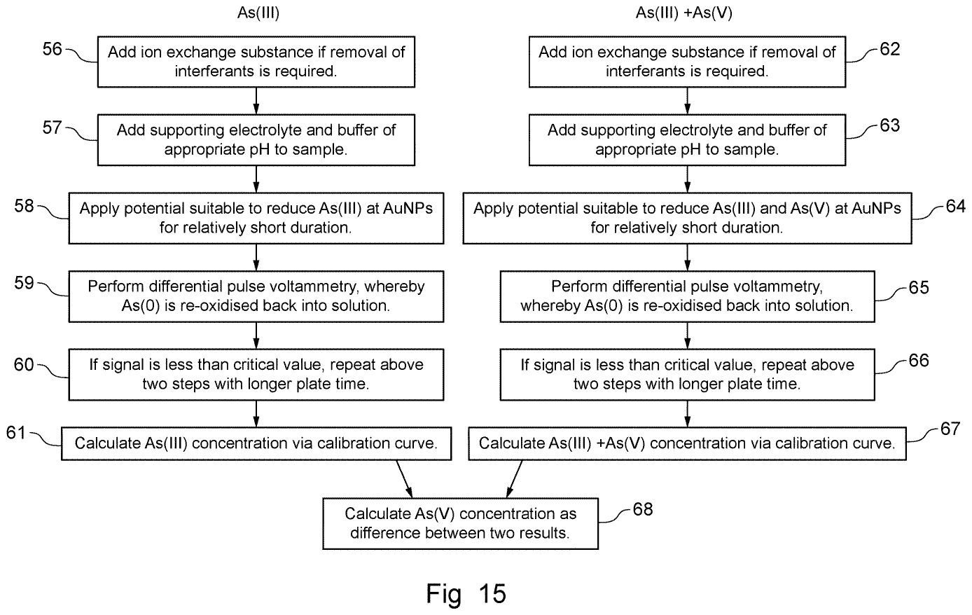

Referring to FIG. 15, the procedure for measuring total arsenic (being the sum of As(III) and As(V)), is similar to the procedure described referring to FIG. 4 but with two differences—the applied potential and the solution pH. As(III) may be electrochemically reduced to As(0) at the AuNP surface, As(V) must first be reduced to As(III) before it may be further reduced to As(0). This is achieved using a substantially more reducing potential (approximately-1.2 V vs. Ag RE) and a lower solution pH, typically around 1. This produces conditions at the surface of the WE 20 that may reduce As(V) to As(III) allowing for subsequent electrochemical reduction to As(0). As(III) is also electrochemically reduced under these conditions and therefore this test is for total arsenic. To calculate the concentration of As(V), the concentration of As(III) was subtracted from that of total arsenic concentration.

In order to process a range of arsenic concentrations, approximately 2-125 ppb, the proposed method employs a dual plate/strip method. Initially, a relatively short plating time of approximately 30 s is conducted by applying the appropriate potential between the working 20 and reference 21 electrodes. DPV is then performed and the stripping current recorded. Using this value, the instrument 11 determines (via firmware and reference libraries 17) whether the test sample belongs to the “high” concentration range or “low” concentration range. In the case of the former, the current value was used to return a concentration via the appropriate calibration curve. In the case of the latter, an additional plating step was conducted at the same potential as before but for a longer duration of approximately 300 s. After this second plating step another stripping step was performed and subsequent calculation of concentration via an appropriate calibration curve. The determination between “high” and “low” range was made by comparing the initial stripping current to a threshold value, based on pre-determined calibration information. According to further implementations and for simplicity both plating steps (short and long) may be conducted regardless of the first result.

In particular, and referring to FIG. 15, to calculate the concentration of As(III) the aqueous sample was passed through an ion exchange column at stage 56 (if removal of interferants or other metal ion species is required). The appropriate supporting electrolyte and buffer of appropriate pH was added to the sample at stage 57. The potential was applied to reduce the As(III) at the coated working electrode 20 for a relatively short time duration at stage 58. Differential pulse voltammetry was performed at stage 59 whereby As(0) was reoxidised back into solution. At stage 60, if a signal (current) measured was less than a predefined value, stages 58 and 59 were repeated with longer preconcentration/plating times. At stage 61, As(III) concentration was calculated via an appropriate calibration curve.

To measure total arsenic concentration (As(III)+As(V)), the aqueous sample was passed through an ion exchange column to remove interference (as required) at stage 62. A supporting electrolyte and buffer, of appropriate pH, was added to the aqueous sample at stage 63. A potential that creates suitable conditions to reduce As(V) to As(III) at the gold coated working electrode 20 was applied for a relatively short time duration at stage 64. Differential pulse voltammetry was performed at stage 65 where As(0) was re-oxidised back into solution. If the observed current was less than a predefined value, stages 64 and 65 were repeated with longer plating/precondition times at stage 66. The concentration of As(III)+As(V) was then calculated via a calibration curve at stage 67. At stage 68, the As(V) concentration was calculated as a difference between the concentrations obtained at stages 67 and 61 as a final stage 68.

Calibration with Temperature Compensation

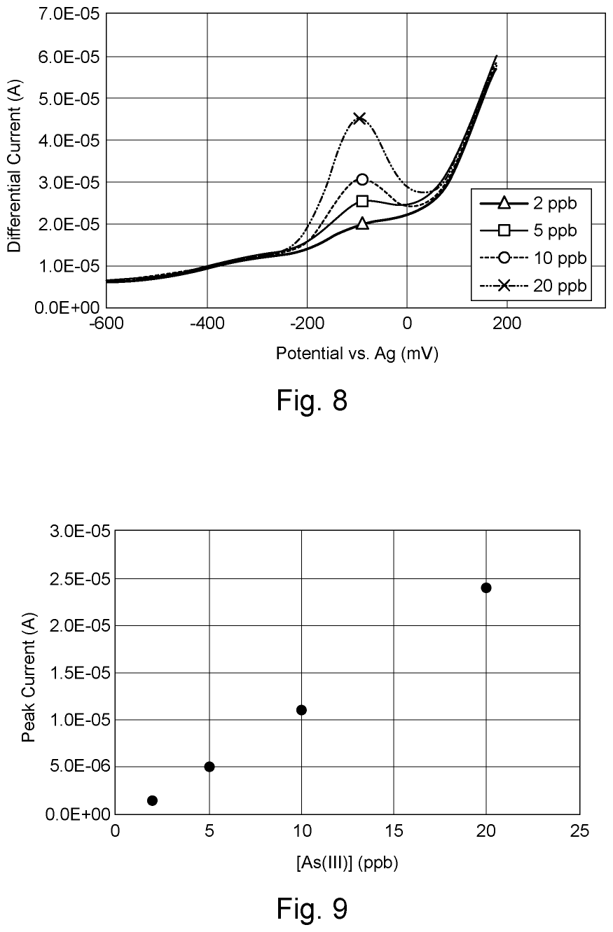

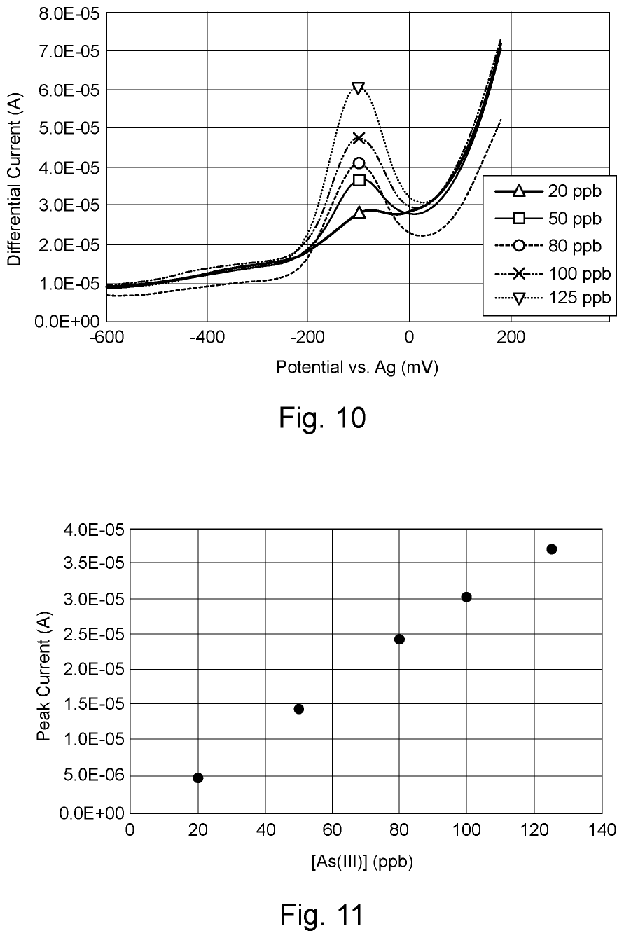

A calibration curve is necessary to relate actual concentrations of arsenic to the signal received when performing ASV with the proposed sensor. The response of a typical sensor to increasing concentrations of As(III) and the measured DPV peak heights plotted against concentration in the low arsenic range are shown below in FIGS. 8 and 9, respectively. Data for the high arsenic range are shown in FIGS. 10 and 11, respectively.

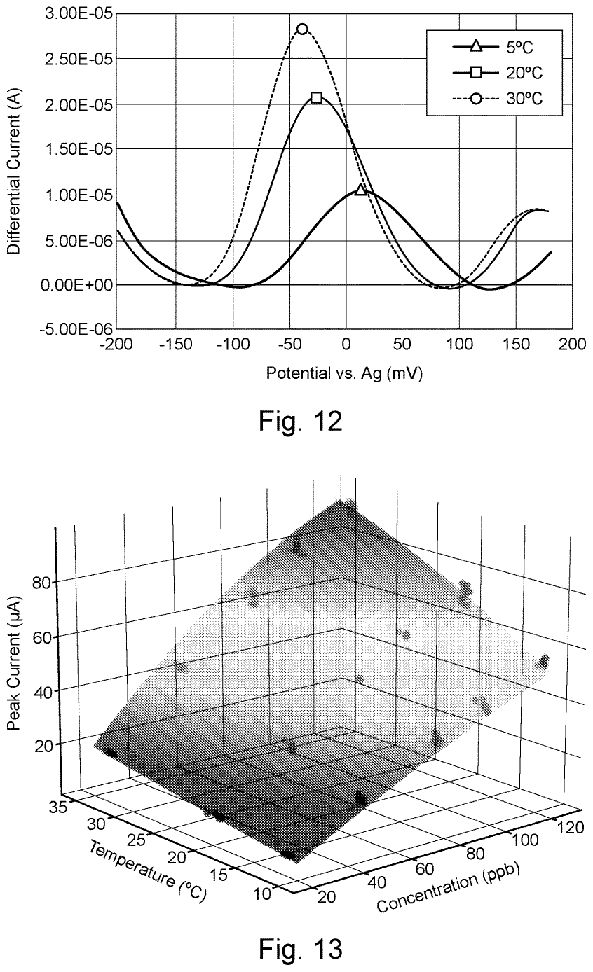

Methods for quantifying analyte concentration may be subject to temperature variation, that is, at different temperatures different signal magnitudes are measured that need to be compensated for. This may be achieved using a lookup table or similar. Electrochemical methods are similarly temperature sensitive with temperature affecting various factors such as diffusion coefficient and electrochemical rate constant. The effect of temperature on the response of a sensor to total arsenic at 20 ppb is shown below in FIG. 12. A difference in both peak magnitude and peak position can be seen as temperature varies.

The present apparatus and method works in conjunction with a temperature measuring element on-board the sensor instrument at sample vessel 15 to make use of a three-dimensional calibration curve. Using the potentiostat 13, (Kemio™ (Palintest Ltd.)), a typical curve generated for this type of calibration is detailed at FIG. 13. This calibration curve enables sensor 19 to correctly relate measured current to arsenic concentration at different temperatures. To generate this curve, systematic measurements are made at a range of arsenic concentrations and solution temperatures and the data fit to a mathematical function using numerical methods. The generated coefficients of this function are then used to calculate concentration from a given temperature and current, i.e. when a user performs a test on a sample of unknown arsenic concentration. A separate calibration curve would be obtained for both As(III) and total arsenic.

### Removal of Interferants

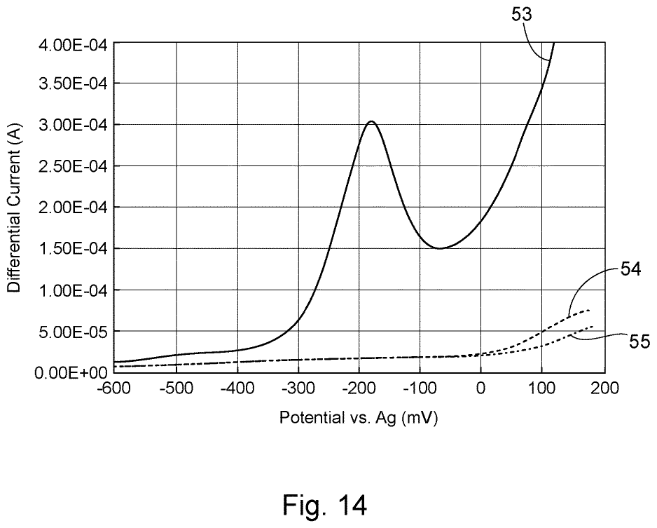

It is known that certain substances may interfere with the proposed method, such as Pb²⁺, Cu²⁺, Cd²⁺ and Fe²⁺/Fe³⁺. Positive interference occurs when the interferant in question is present at or suitably close enough to the stripping potential of arsenic, thereby giving an erroneously high reading because the signals cannot be deconvoluted. Arsenic typically does not form cationic species (Determination of arsenic species: A critical review of methods and applications, 2000-2003, Kevin A. Francesconi and Doris Kuehnelt), therefore interferants present as cations may be removed via ion exchange without reducing the concentration of arsenic compounds Typically, cationic exchange resins are employed that comprise crosslinked polymers with appropriate functional groups for chelation. Examples include Puromet MTS9300 and Puromet MTS9500 from Purolite, DIAION CR11 from Mitsubishi Chemical Corporation and Dionex™ OnGuard™ II M from Thermo Scientific. Arsenic generally exists as Arsenite (AsIII) and Arsenate (AsV) that are anions, so they do not bind to the cation exchange column. The effect of cation removal with ion exchange is shown below in FIG. 14. The lowest dashed line 55 is simply that of a blank solution, i.e. supporting electrolyte and buffer. The solid line 53 shows a solution mixture comprising 2000 ppb of Cu²⁺, 50 ppb of Pb²⁺ and 20 ppb of Cd²⁺, the upper dashed line 54 represents the same solution after treatment by passing through the cation exchange column. Prior to treatment with the ion exchange material, a prominent peak is present at approximately −180 mV. This is close enough to the expected peak location of arsenic (−100 mV) to cause erroneously high readings. In addition, a large feature is observed at more positive potentials that rises continually. This feature is largely mitigated after the addition of ion exchange beads as can be seen by dashed line 54.

### Signal Normalisation

The proposed sensor comprises an SPCE electrode 20 that is augmented by AuNPs for the detection of different forms of arsenic in water samples. The signal produced by the sensor is a function of not only arsenic concentration and solution temperature but also AuNP loading onto the carbon substrate. As with any manufacturing process, small deviations occur between sensors produced using drop casting, creating different signal efficiencies. Sensors that have a higher quantity of AuNPs return a higher signal on average, and the reverse is true for sensors with a lower quantity of AuNPs. To compensate for this, the present method utilises signal normalisation, whereby the signal obtained during the arsenic stripping phase is divided by the signal obtained during the plating phase. A higher-than-average plating current is observed (at a given concentration and temperature) when a sensor has a higher quantity of AuNPs. This extra signal is effectively used to eliminate extra signal produced during the stripping phase. Tables 1 and 2 illustrate a decrease in the Relative Standard Deviation (RSD) of two different sensor batches upon implementation of signal normalisation for total arsenic.

TABLE 1

| Plating current Sum | Peak Height | Peak Height/ |
|---|---|---|
| (A) | (A) | Plating Current Sum |
| 1.60E−01 | 6.23E−05 | 3.90E−04 |
| 1.71E−01 | 6.19E−05 | 3.62E−04 |
| 1.63E−01 | 5.99E−05 | 3.68E−04 |
| 1.67E−01 | 5.90E−05 | 2.53E−04 |
| 1.53E−01 | 5.86E−05 | 3.83E−04 |
| 1.56E−01 | 5.75E−05 | 3.69E−04 |
| 1.55E−01 | 5.75E−05 | 3.71E−04 |
| 1.61E−01 | 5.75E−05 | 3.56E−04 |
| 1.57E−01 | 5.81E−05 | 3.70E−04 |
| 1.71E−01 | 6.14E−05 | 3.58E−04 |
| 1.64E−01 | 6.14E−05 | 3.75E−04 |
| 1.74E−01 | 6.24E−05 | 3.58E−04 |
| 1.52E−01 | 5.75E−05 | 3.77E−04 |
| 1.55E−01 | 5.69E−05 | 3.66E−04 |
| Mean | 5.94E−05 | 3.68E−04 |
| Standard Deviation | 2.05E−06 | 1.06E−05 |
| Relative Standard Deviation | 3.46% | 2.87% |

TABLE 2

| Plating current Sum | Peak Height | Peak Height/ |
|---|---|---|
| (A) | (A) | Plating Current Sum |
| 1.67E−01 | 5.99E−05 | 3.59E−04 |
| 1.72E−01 | 6.11E−05 | 3.55E−04 |
| 1.68E−01 | 6.05E−05 | 3.60E−04 |
| 1.75E−01 | 6.23E−05 | 3.56E−04 |
| 1.62E−01 | 5.72E−05 | 3.53E−04 |
| 1.69E−01 | 5.97E−05 | 3.53E−04 |
| 1.62E−01 | 5.88E−05 | 3.62E−04 |
| 1.70E−01 | 6.03E−05 | 3.55E−04 |
| 1.72E−01 | 6.22E−05 | 3.62E−04 |
| 1.72E−01 | 6.09E−05 | 3.55E−04 |
| 1.62E−01 | 5.81E−05 | 3.59E−04 |
| 1.71E−01 | 6.15E−05 | 3.59E−04 |
| 1.62E−01 | 5.83E−05 | 3.60E−04 |
| 1.77E−01 | 6.23E−05 | 3.53E−04 |
| Mean | 6.02E−05 | 3.57E−04 |
| Standard Deviation | 1.64E−06 | 3.16E−06 |
| Relative Standard Deviation | 2.73% | 0.88% |

1. An electrochemical method for the analysis of an analyte in a liquid sample comprising:
performing electrochemical stripping voltammetry on a liquid sample using a potentiostat and a cell with working, reference and counter electrodes involving electrodeposition followed by oxidation of the analyte at the working electrode and measurement of current to determine an analyte peak current during the oxidation of the analyte;
measuring a charge or current at the working electrode during the electrodeposition of the analyte at the working electrode; and
normalising the peak current determined during the oxidation of the analyte by applying at least one mathematical operation on said peak current involving a division of the peak current by the charge or current measured during electrodeposition.

2. The method as claimed in claim 1 wherein the step of performing electrochemical stripping voltammetry comprises applying a plating potential to the working electrode and acquiring plating current data measured at the working electrode during the application of the plating potential.

3. The method as claimed in claim 2 further comprising storing the plating current data at a data storage utility.

4. The method as claimed in claim 2 comprising applying a stripping potential to the working electrode and acquiring stripping current data measured at the working electrode during the application of the stripping potential.

5. The method as claimed in claim 4 further comprising storing the stripping current data at a data storage utility.

6. The method as claimed in claim 1 wherein the step of applying the mathematical operation consists of division of the peak current of the oxidation of the analyte by the charge or current measured during electrodeposition.

7. The method as claimed in claim 1 wherein the analysis comprises the quantitative concentration analysis of the analyte in the sample by applying a mathematical function module to the normalised peak current to generate a normalised concentration of the analyte in the sample.

8. The method as claimed in claim 7 wherein the mathematical function module is generated from a mathematical fitting of known analyte concentration data and corresponding reference peak current data measured using electrochemical stripping voltammetry during a concentration-current calibration process.

9. The method as claimed in claim 8 wherein the peak current reference data is normalised comprising the step of during the electrodeposition of the analyte at the working electrode, measuring a charge or current at the working electrode and normalising the peak current determined during the oxidation of the analyte by dividing the peak current by the charge or current measured during electrodeposition.

10. The method as claimed in claim 1 wherein prior to the step of performing the electrochemical stripping voltammetry, removing metal cation interferants from the sample involving contacting the sample with a cation exchange component.

11. The method as claimed in claim 10 wherein the cation exchange component comprises a cation exchange column and the step of contacting the sample comprises passing the sample through the cation exchange column and collecting filtered sample.

12. The method as claimed in claim 1 wherein the method further comprises:
measuring a temperature of the sample at least during the oxidation of the analyte; and
applying the measured temperature and the peak current to a mathematical function module derived from mathematical fitting of reference peak current data, reference temperature data and reference analyte concentration data measured and determined during electrochemical stripping voltammetry as part of a temperature-concentration-current calibration process to determine a temperature calibrated concentration of the analyte in the sample.

13. The method as claimed in claim 12 wherein the temperature-concentration-current calibration process comprises measuring the temperature of a reference sample containing a known concentration of analyte simultaneously with the measuring of the charge.

14. The method as claimed in claim 1 wherein the analyte comprises a metal analyte and optionally wherein the metal analyte comprises any one of: arsenic, lead, cadmium and copper.

15. The method as claimed in claim 1 wherein the electrochemical stripping voltammetry comprises any one of anodic, cathodic and adsorptive stripping voltammetry.

16. Apparatus for the electrochemical analysis of an analyte in a liquid sample comprising:
a sensor having working, reference and counter electrodes configured to be at least partially submerged in an electrolyte solution containing a analyte;
a potentiostat electrically connectable and configured to apply a potential to the working electrode; and
a control utility having a normalisation module to receive charge or current data measured at the working electrode during an electrodeposition of the analyte at the working electrode and to receive current data measured during an oxidation of the analyte at the working electrode and to calculate a normalised peak current of the oxidation of the analyte at the working electrode.

17. The apparatus as claimed in claim 16 further comprising an electrochemical potential module to generate and apply a potential difference between the working and reference electrode.

18. The apparatus as claimed in claim 17 wherein the electrochemical potential module comprises a plating module to generate and apply a potential difference between the working and reference electrode and deposit analyte at the working electrode.

19. The apparatus as claimed in claim 17 wherein the electrochemical potential module comprises a stripping module to generate and apply a pulsed potential to the working electrode and to oxidise the analyte at the working electrode.

20. The apparatus as claimed in claim 16 further comprising a data acquisition module to receive charge or current data during an electrodeposition and oxidation of the analyte at the working electrode.

21. The apparatus as claimed in claim 16 wherein the normalisation module is configured to perform a mathematical operation on the peak current data acquired during oxidation of the analyte at the working electrode comprising division on the peak current data by the charge or current data acquired during electrodeposition of the analyte at the working electrode to generate the normalised peak current of oxidation of the analyte at the working electrode.

22. The apparatus as claimed in claim 21 further comprising a mathematical function module configured to process the normalised peak current data and to generate an analyte concentration.
